(19) United States
(12) Patent Application Publication
(10) Pub. No.: US 2012/0082687 A1
(43) Pub. Date: Apr. 5, 2012

(54) Use of cell adhesion inhibitor for the mobilization of antigen presenting cells and immune cells in a cell mixture (AIM) from the peripheral blood and methods of use

(76) Inventors: Alex Wah Hin Yeung, Newport Beach, CA (US); Katie Jessica Yeung, Newport Beach, CA (US)

(21) Appl. No.: 12/924,698

(22) Filed: Oct. 4, 2010

(51) Int. Cl. A61K 35/12; A61P 37/04; A61P 31/18; A61K 39/00; A61P 35/00

(52) U.S. Cl. 424/184.1; 424/93.71

(57) ABSTRACT

Disclosed is a method to recover an antigen presenting cells (APCs) and immune cells rich mixture (AIM) from peripheral blood mononuclear cells (PBMC) mobilized with one or more cell adhesion inhibitors for the preparation of an AIM vaccine or an AIM adoptive immunotherapy preparation. In addition, AIM mobilization can be enhanced by priming, simultaneously or in sequence, one or more of a combination of different chemical compounds, cytokines, hormones, growth factors, etc. The interaction of chemokines and chemokine receptors enable tumor cells attachment or in close proximity to antigen presenting cells and immune cells which possess similar receptors in a micro niche environment. Severing the chemokine/chemokine receptor linkage by a cell adhesion inhibitor will release these specifically primed cell mixtures into the peripheral blood. The collection of these cells from the peripheral blood has never been described and is the basis of this invention. AIM cells can either be used alone or better still, be induced into more target specific preparations with additions, modifications and incubation, pre or post cell adhesion inhibitors mobilization, with vaccines, different target specific antigens, peptides, chemotherapeutic agents, oncolytic viral therapeutic agents, cytokines, co-stimulatory molecules, anti-regulatory T cell therapeutic agents, anti-CTLA4, anti-PD1 molecules and other methodologies of immunological enhancement known to the art. The AIM vaccine or AIM adoptive immunotherapy preparation can then be used, but not limited to, the treatment of cancer and other diseases.

### BACKGROUND OF THE INVENTION

Peripheral blood derived mononuclear cells usually are mobilized by growth factors acting on the bone marrow. The use of G CSF and GM CSF as mobilization agents for autologous stem cell support after high dose chemotherapy dated back nearly twenty years during the time when such therapy was popular with lymphomas and chemosensitive solid tumors such as late stage breast cancer (Yeung 1994).

Cell adhesion inhibitors such as Plerixafor, a macrocyclic compound antagonist of the alpha chemokine receptor CXCR4, was approved by the FDA in late 2008 for hematopoietic stem cell (HSC) mobilization. The SDF-1/CXCL12/CXCR4 retention axis disruption by this agent in the bone marrow can release a whole host of progenitor cells without the necessity of priming. The result in HSC collection using G CSF and Plerixafor has been dramatic. Little, if any, is known of the effect of CXCR4 antagonist such as Plerixafor on the mobilization of AIM cells or dendritic cells (DC) precursors into the peripheral blood. Even though DCs are the pivotal antigen presenting cells (APC), the term APC is used somewhat interchangeably with DC for the remaining description of this invention. As shown later, the present invention will encompass the collection of a whole spectrum of other cells such as immune T, B and NKT cells and other non dendritic antigen presenting cells in a mixture, and hence the terms APCs for antigen presenting cells and AIM for the combination of APCs and immune cells are now more appropriate.

The use of Plerixafor or other cell adhesion inhibitors for the mobilization and collection of APCs and AIM from peripheral blood has never been described. DCs, the major player in the APC spectrum, express cell adhesion receptors such as CCR1 to CCR5, CXCR2 and 4 in the precursor and immature stage, and CXCR4 and 5 in the mature stage (Oppenheim 2002). The stroma derived factor 1 (SDF-1 or CXCL12) bound exclusively to CXCR4 (with the rare exception of CXCR7) and SDF-1 is a factor expressed by a majority of cancer cells (Kryczek 2007). Besides improving the proliferation and survival of the tumor cells, SDF-1 also enhances vascular supply and new blood vessel formation. Consequently tumors that expresses SDF-1 will be able to grow while attracting a variety of immune cells including DCs. The general immune suppressing environment, created from myeloid suppressor, tumor associated macrophages and T regulatory cells will in most cases suppress the activities of DCs, rendering them into a resting and anergic state. There is, however, a population of “poised” T cells (central memory like CD8+ cells) that can be revived by TLR ligands, LPS or CD40 agonist monoclonal antibody (Melief 2008).

Because of the general acceptance that most cancer cells express CXCR4 receptors, the use of Plerixafor as a cell adhesion inhibitor in the SDF-1/CXCR4 axis will be able to disrupt this axis and therefore release into the peripheral blood a collection of immature as well as mature DCs that have come into contact and possible activation with tumor cells. Furthermore, as other immune cells such as CD4+ and CD8+ T cells, B cell and NKT cell also express CXCR4 and/or linked to the same SDF-1/CXCR4 axis, a mixture of these immune cells will also be mobilized into the peripheral blood. It is with this understanding that AIM may be collected and used by the method described in this invention.

Dendritic or APC based vaccines or adoptive immunotherapy preparations have been known to the art for over 20 years and most of them derived their source of DCs from the peripheral blood mononuclear cells. Besides, numerous clinical trials have demonstrated the safety of APC based vaccines, and literally thousands of patients have received some form or another of APC vaccines with no serious adverse events. Notable clinical responses, though not always easy to come by, have been observed in about one tenth of the patients (Rideway 2003) (Banchereau 2001). A recent study showed that vaccination using APCs loaded with four melanoma peptides (gp100, melan-A/MART-1, tyrosine melanoma antigen (MAGE-3), KLH and flu matrix resulted in regression of metastatic melanoma after four bimonthly vaccinations (Banchereau 2001). Another study for prostatic cancer, though not be able to demonstrate the intended delay of disease progression, demonstrated significant increase in median survival (Small 2007).

APC plays a crucial role in the stimulation of primary and secondary CD4+ and CD8+ T cell responses. Immature APCs are characterized by more efficient phagocytic activity for antigen up-take and processing within the cells. During the maturation process, these cells become less efficient in antigen capturing but more specialized in presenting immunogenic peptides and in activating naive T cells. For an APC based immunotherapy to be successful, a combination of immature and mature cells and their synergistic action may be useful. APC, especially dendritic cells (DC), maturation can be mediated by inflammatory cytokines, or by additional stimuli such as CD40L, LPS or virus infection. All these stimuli add up to a rapid and sustained up-regulation of MHC class I antigen-processing machinery as well as of co-stimulatory molecules (CD40, CD80, CD86) and DC maturation marker CD83. Combination of these activation and co-stimulatory molecules is necessary for T-cell activation and the generation of cytotoxic CD8+ cells for the treatment of cancer.

During a viral infection or a malignant transformation, APCs acquire antigens from the affected sites. These cells then migrate to the draining lymph nodes and present peptides associated MHC class I molecules, to helper CD4+ cells and CD8+ T cells. The way by which DCs phagocytose these foreign antigens from the extracellular environment and effectively present those selected peptides associated to MHC class I molecules, to CD8+ T cells, is called cross-presentation and is likely the most important mechanism for the priming of CD8+ T cells responses against foreign antigens.

The priming and expansion of antigen-specific CD8+ T cell response is a complex process involving concerted interactions between lymphocytes and other professional antigen-presenting cells. This process plays an important role in linking innate and adaptive immunity. The priming of antigen-specific CD8+ T cells requires recognition through the T cell receptor of peptide-MHC class I complexes on the surface of appropriate APCs, as well as feedback mechanisms and interaction with the regulatory and suppressor cell system.

However, suitable peptides may also be derived from exogenous antigens intersecting this pathway after endocytosis by APCs, in the cross-presentation process. DCs must undergo a special activation process or confirmation step in order to cross-prime CD8+ T cells (Blankenstein 2002). Cross-presentation of antigens by unconfirmed DCs may stimulate an abortive response that culminates in cross-tolerance (Belz 2002). Under pathological conditions, DCs are confirmed by engagement of surface CD40 by activated CD4+ helper T cells or by viral derived macromolecules, which can trigger DC maturation and up regulate and thereby increase the expression of different surface co-stimulatory molecules such as the most fundamental CD28/CD80, CD86 co-stimulatory signal number 2.

It has been reported that only mature DCs, such as those obtained from culturing immature GM-CSF/IL-4 DCs with tumor necrosis factor (TNFalpha) and prostaglandin E2, are efficient antigen presenting cells (APCs) for cross-priming of exogenous antigens to CD8+ T cells (Schuler 2003) (Kaiser 2003).

Other studies demonstrated that DCs generated from human peripheral blood mononuclear cells after a single-step 3-days culture in the presence of IFN-alpha and GM-CSF, exhibit phenotypic and functional properties typical of activated partially mature DCs (Santini 2000). It is interesting to know that IFN induced DCs 133 are more efficient than immature DCs, in inducing a Th-1 type immune response and CD8+ T cells response against defined antigens in a variety of models (Lapenta 2003) (Santodonato 2003) (Gabriele 2004) (L. a. Santini 2006). In this scenario, IFN-alpha, a well known drug, may be one of the compounds that can be used, in addition to TNF alpha and prostaglandin E2, for the final maturation and cross priming of CD8+ cells.

In addition, it has generally been assumed that CD34+ derived mature DC is better than those coming from committed mononuclear cells. These CD34+ derived mature DC can efficiently induce cross-priming of CD8+ T cells against exogenous antigens (LeBon 2002) (Blankenstein 2002) and can easily be mobilized using the standard G-CSF protocol.

So, as a summary of the current state of the art preparation of a DC vaccine from PBMC or otherwise, the first step is to culminate immature DCs which are active in phagocytic activity from several days of culture in the presence of GM-CSF/IL-4.

The second culture step, with the addition of activation factors such as IFN, TNF alpha and prostaglandin E2, more mature DCs that are capable of cross priming are generated (LeBon 2002) (Ridge 1998).

The third step is to incorporate the designed antigen or antigen inducing methodology into this DC vaccine preparation with or without the further addition of an adjuvant.

This invention is to improve on the current state of the art preparation of DCs or APCs vaccine and adoptive immunotherapy based on the same type of cells by an unique process that only requires the use of one or more than one cell adhesion inhibitor molecules before the mobilization and collection of peripheral blood mononuclear cells, containing APCs and immune cells mixture (AIM). It is, however, important to note that different methodologies, either pre or post the mobilization and collection process of AIM, will enhance the success of such therapy. Some of these methodologies will be described.

Pre mobilization and collection of AIM enhancement technology may include, but not limited to, the use of vaccines, oncolytic viruses, cell based therapies, cytokines that may be given systemically or being expressed by vaccines, oncolytic viruses or other transduction methodologies, chemotherapy and other procedures that are well known to those familiar with the art.

Post mobilization and collection of AIM enhancement technology may include ex vivo manipulation or incubation with different forms of vaccines, cytokines, anti-CTLA4, anti-CD25 and anti PD-1 molecules or antibodies, CD40 agonists, LPS, TLR ligands, and other methodologies that would either enhance the Th1 process, or would be able to bypass or reduce the amount of immuno-suppression in the generation of cytotoxic T lymphocytes.

Glossary: (in the Context Applied to this Invention)

Effective dose range: An effective dose in pharmacology is the amount of drug that produces a therapeutic response in 50% of the people taking it, sometimes also called ED-50. An effective dose range described in this invention means a dose range that is from 0.01% to 100 times the effective dose normally used. This effective dose range definition also extends to ex vivo culturing of AIM.

### Group A:

Cytokine and immune related agents: G-CSF, GM-CSF, IL4, IFN, SCF (Steele factor, Stem Cell Factor kit ligand), TNF alpha (tumor necrosis factor alpha), prostaglandin E2, IL1, IL6, CD40L

### Group B:

### Anti Down-Regulate or Up-Regulate Immune Cell Agents:

Anti-CTLA 4: CTLA 4 (CD152) is a member of the immunoglobulin superfamily, which is expressed on the surface of Helper T cells and transmits an inhibitory signal to T cells. CTLA4 is similar to the T-cell costimulatory protein CD28, and both molecules bind to CD80 and CD86 on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules (i.e. CD80 and CD86).

Multiphoton microscopy studies observing T-cell motility in intact lymph nodes gave evidence for the so called ‘reverse-stop signaling model’. In this model CTLA 4 reverse the classic TCR-induced ‘stop signal’ needed for firm contact between T cells and antigen-presenting cells (APCs).

Anti-PD1: Programmed Death 1, or PD-1, is a Type I membrane protein of 268 amino acids. PD-1 is a member of the extended CD28/CTLA-4 family of T cell regulators. The protein's structure includes an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates TCR signals. This is consistent with binding of SHP-1 and SHP-2 phosphatases to the cytoplasmic tail of PD-1 upon ligand binding. PD-1 is expressed on the surface of activated T cells, B cells, and macrophages, suggesting that compared to CTLA-4, PD-1 more broadly negatively regulates immune responses.

CD 40 agonist (CD154): CD40 is a costimulatory protein found on antigen presenting cells and is required for their activation. The binding of CD154 (CD40L) on T_{H} cells to CD40 activates antigen presenting cells and induces a variety of downstream effects.

LPS: Lipopolysaccharides, also known as lipoglycans, are large molecules consisting of a lipid and a polysaccharide joined by a covalent bond; they are found in the outer membrane of Gram-negative bacteria, act as endotoxins and elicit strong immune responses in animals.

TLR ligands: Ligands that activate toll-like receptors (TLRs) which are a class of proteins that play a key role in the innate immune system. They are single, membrane-spanning, non-catalytic receptors that recognize structurally conserved molecules derived from microbes. Once these microbes have breached physical barriers such as the skin or intestinal tract mucosa, they are recognized by TLRs which activates immune cell responses.

### Group C:

### Anti Treg and Other Suppressors of Immune Cells Agents:

Chemotherapeutic agents: Chemotherapy, in its most general sense, is the treatment of a disease by chemicals especially by killing micro-organisms or cancerous cells. In popular usage, it refers to antineoplastic drugs used to treat cancer or the combination of these drugs into a cytotoxic standardized treatment regimen.

Anti-CD25: CD25 is the alpha chain of the IL-2 receptor. It is a type I trans-membrane protein present on activated T cells, activated B cells, some thymocytes, myeloid precursors, and oligodendrocytes that associates with CD122 to form a heterodimer that can act as a high-affinity receptor for IL-2.

GITR agonist: GITR (glucocorticoid-induced tumor necrosis factor receptor) is a surface receptor molecule that has been shown to be involved in inhibiting the suppressive activity of T-regulatory cells and extending the survival of T-effector cells.

OX 40 agonist: a co-stimulatory molecule belonging to the TNF receptor super family that leads to expansion of CD4+ and CD8+ T cells

Corticosteriods: known hormones or known classes of synthetic molecules that will reduce immune cell activities, especially those associated with suppressor cells.

Priming: (in the Context of this Invention)

Priming means any form of a therapy, by itself or in combination with another agent, that is being administered to an individual for AIM therapy before the apheresis process. It is distinct from the cell adhesion inhibitor/s given prior to apheresis, which is an essential and non-alienable part of this invention. A typical priming procedure in this invention may be the administration of cytokines such as GM-CSF or IFN for a few days or, a cell or peptide vaccine given by an intra dermal route or an oncolytic virus given by an intra tumor route (with or without gene expressions such as GM-CSF or IFN) before cell adhesion inhibitor and apheresis for AIM cells. As described in other examples shown later, priming in this invention may mean a combination of many other forms of therapy and treatment, all done prior to aphereis.

### Procedures and Methods:

### Apheresis or Leukapheresis for AIM:

Vascular access is accomplished by an antecubital venipuncture, or if necessary, central venous lines with a double or single human catheter (depending on the blood cell separator machine being used). The procedure is accomplished by a single peripheral vein and to adapt the software to accommodate an intermittent, or discontinuous flow procedure following the same protocol used for mononuclear cell collection. For example, AIM can be collected on a Fenwal CS-3000 continuous flow blood cell separator (Fenwal Laboratories, Deerfield, Ill.). Closed system apheresis software, with pre-attached transfer packs, 1 litre bags of both 0.9% sodium chloride and acid citrate dextrose, and two butterfly needles, are utilized to reduce the risk of bacterial contamination to the patient and to the cells collected. Red blood cells, leukocytes, platelets, and plasma are returned to the patient through another antecubital line. After about 10 litres of peripheral blood are processed, the contents of the collection container consisted of 200 cc of plasma plus platelets and of a pellet containing platelets and mononuclear cells. The pellet is re-suspended by manual agitation and centrifuged for 3 minutes at 100 rpm to separate the platelet rich plasma from the mononuclear pellet. The platelet rich plasma is siphoned into a clean transfer pack and re-infused to the patient. AIM cells that remained in the collection bag are then processed using a closed, automated protocol. The cells are re-suspended by agitation without the addition of saline. Three hundred ml of Ficoll are aseptically transferred to a 600-ml transfer pack and connected to the apheresis software via the injection site on the component rich plasma line. Ficoll is underlaid at 4 ml/min without centrifugation to establish a density gradient until the collection container is full. The centrifuge is re-started and the supernatant plasma and platelets are collected into a waste bag until the cells appeared in the supernatant line. AIM cells are then diverted into an empty transfer pack, and the processing is continued until all of the Ficoll is used. The residue in the collection bag is washed out and directed into a waste bag. The purified AIM are re-suspended in 400 ml of normal saline, returned to the collection bag within the centrifuge chamber, and washed with another 1 litre of normal saline. The AIM is then re-suspended into 200 ml and transferred to a cell culture flask or a sterile final product bag.

AIM Ex-Vivo Culture Methodology: (Reagents, Media, Conditions and Length of Culture etc. Vary with Different Protocols and the Description Below is Just One of Many Examples to Use AIM Cells and Should Not be Construed as the Only Way to Practice this Invention)
Protocol 1: AIM cells only

Prepare a medium for the culture of human PBMC: supplement 500 ml RPMI medium with 2% human AB serum, 2 mM L-glutamine, 50 U/ml penicillin and 50 μg/ml streptomycin (complete medium).

Count the AIM cells and re-suspend them in complete medium at a concentration of 0.25 to 4.5 million cells per 100 ml. This cell concentration may vary with different protocols.

Incubate the cells for 4 to 24 hours at 37° C. and 5% CO₂.

AIM cells are now ready to be given as aliquots for vaccine (some alliquots will be cryo-preserved for future vaccination) therapy or as a preparation to be given systemically for adoptive immunotherapy.

Protocol 2: AIM Cells Incubation with Group B and/or Group C Agents with or without Loading of Specific Antigens (Tumor Vaccine, Peptides etc.)

Prepare a medium for the culture of human PBMC: supplement 500 ml RPMI medium with 2% human AB serum, 2 mM L-glutamine, 50 U/ml penicillin and 50 μg/ml streptomycin (complete medium).

Count the AIM cells and re-suspend them in complete medium at a concentration of 0.25 to 4.5 million cells per 100 ml. This cell concentration may vary with different protocols.

Add Group B and/or Group C agents at the “effective dose range” of each of the components with or without the loading of specific antigens such as irradiated tumor cells, peptides or DNA vaccines etc.

Incubate the cells for the time period ranging from 4 hours to 48 hours at 37° C. and 5% CO₂. Incubation time varies with different protocols known to the art. Depending on the length of the incubation period, proper quality control in regards to contamination, viral and bacterial infestation, toxins, cell counts and characteristics have to be performed before the final product is ready for human use. A longer period of incubation and expansion (up to 7 days or beyond) may be necessary for some agents.

After passing the necessary quality control procedures, the ex vivo modified AIM cells are now ready to be given as aliquots for vaccine (some aliquots will be cryo-preserved for future vaccination) therapy or as a preparation to be given systemically for adoptive immunotherapy.

Protocol 3: AIM Cells Incubation with Group A Agents such as GM-CSF and Pro-Inflammatory Mediators before Loading of Specific Antigens With or Without Group B and/or Group C Agents

Prepare a medium for the culture of human PBMC: supplement 500 ml RPMI medium with 2% human AB serum, 2 mM L-glutamine, 50 U/ml penicillin and 50 μg/ml streptomycin (complete medium).

Count the AIM cells and re-suspend them in complete medium at a concentration of 0.25 to 4.5 million cells per 100 ml. This cell concentration may vary with different protocols.

Add 500 U/ml IL-4 and 1000 U/ml GM-CSF or other effective dose range to the cell suspension.

Incubate the cells for 24 to 96 hours at 37° C. and 5% CO₂. Incubation time varies with different protocols known to the art.

After that add a cocktail of pro-inflammatory mediators to the cell suspension: 1000 U/ml TNF-α, 10 ng/ml IL-1β, 10 ng/ml IL-6 plus 1 μM PGE₂. Other effective dose range may apply.

Incubate the cells again for 24 to 96 hours at 37° C. and 5% CO₂. Incubation time varies with different protocols known to the art.

After this incubation, load specific antigens such as irradiated tumor cells, peptides, DNA vaccines etc, with or without the addition of Group B and/or Group C agents at the “effective dose range” of each of the components.

Incubate the cells for the time period ranging from 4 hours to 48 hours at 37° C. and 5% CO₂. Incubation time varies with different protocols known to the art. Depending on the length of the incubation period, proper quality control in regards to contamination, viral and bacterial infestation, toxins, cell counts and characteristics have to be performed before the final product is ready for human use. A longer period of incubation and expansion (up to 7 days or beyond) may be necessary for some agents.

After passing the necessary quality control procedures, the ex vivo modified AIM cells are now ready to be given as aliquots for vaccine (some alliquots will be cryo-preserved for future vaccination) therapy or as a preparation to be given systemically for adoptive immunotherapy.

Vaccines: (In this Context it Includes Different Therapies that may Evoke a Specific Tumor Immunotherapeutic Response)

Irradiated autologous or allogeneic tumor cells, tumor lysates or apoptotic tumor cells.

Known tumor specific or associated antigens or peptides such as Mage-1, Mage-3, gp-100 and MUC-1 etc.

Dendritic cell tumor hybrids generated by electrofusion or polyethylene glycol, gene insertion with tumor-associated antigen (TAA), tumor derived mRNA etc.

Modified autologous or allogeneic cell vaccines that express different genes and combination of gene products such as GM-CSF in GVAX.

Oncolytic viral therapies that express gene and combination of gene products, e.g. cytokines such as GM-CSF or type 1 IFN and those that selectively proliferates only in certain tumor types such as PSA in prostate cancer etc.

Vectors and methods known to the art that can express gene and a combination of gene products that have the effect of evoking specific and non specific tumor immunotherapy in an individual.

### SUMMARY OF THE INVENTION

This invention, even though it is a mobilization methodology of specific cell types into the peripheral blood, specifically differs from the traditional practice by not employing G-CSF as the sole mobilization agent. In addition, this invention will not employ the cell adhesion inhibitor for the purpose of collecting hematopoietic progenitors for bone marrow rescue in the treatment of multiple myeloma or the lymphomas.

The present invention provides a platform of harvesting, after mobilization, a special mixture of cells containing antigen presenting cells (APCs) such as the different maturity types of dendritic cells (DCs) and immune cells consisting of CD4+, CD8+ T cells and NKT cells (AIM) from peripheral blood mononuclear cells (PBMC) by administering to an individual with one or a combination of more than one cell adhesion inhibitors such as Plerixafor. Because of the nature of the conditions under which the ligands are attached to the cell adhesion receptors linking cancer cells with the stroma as well as cell types that later developed into AIM cells, it is expected that many of the cell types and cell status within the AIM preparation are activated and proliferation prone in the generation of a specific adaptive response or a combination of adaptive responses towards the cancer cells. These AIM cells are mobilized after that link, as described, has been severed by the cell adhesion inhibitor/s. They will transverse into the peripheral blood and be collected by the different methodologies known to the art, one of which is by apheresis with leukapheresis of the mononuclear fraction by a commercially available blood cell separator. By fine-tuning this response towards a particular cell type or antigen with other enhancement techniques pre and post mobilization, either in or ex vivo, AIM vaccine or preparation may be effective in the treatment of cancer or infectious diseases.

The unique concept to physically disrupt the link between cancer, or other target cells, in the micro niche environment with either the stromal cells or with the antigen presenting cells and immune cells (which specifically are also attracted to the same micro niche environment by the presence of chemokines) by cell adhesion inhibitors and to apply that to the practice of tumor immunotherapy has never been described previously in the literature.

### DETAIL MODES FOR CARRYING OUT THE INVENTION

Our invention is a discovery of a new process from which a specially useful mixture of cell types (AIM) that include APCs (such as all types of DCs) and other immune cells (such as CD4+, CD8+ T cells, NKT cells etc.) after they are mobilized into the peripheral blood by the use of one or a combination of more than one cell adhesion inhibitors. The AIM cells can then be collected in the PBMC fraction during apheresis. By the nature of the cell adhesion inhibitor/s used in this process, some, or all of these cells may be primed, activated or poised for proliferation and be used as adoptive immunotherapy in the fight against cancer or other infectious diseases. Since cancer immunotherapy is a complicate process in which most of the human cancer cells should have already acquired a state of self-tolerance, trying to break this tolerogenicity sometimes require ex vivo manipulation of specific cell types with agents, chemicals or antibodies that may be quite toxic if given systemically. This invention can be a first step to bring out some of the key elements of cellular adaptive immunotherapy into an ex vivo process for fine-tuning so that an effective cancer immunotherapy can be carried out without causing an excessive auto immune response.

In a first aspect, the invention describes a method of preparation of a unique population of cell adhesion inhibitor mobilized AIM cells derived from the peripheral blood mononuclear cells (PBMC) by the administering to an individual with one or a combination or more than one cell adhesion inhibitors such as Plerixafor (a CXCR4 antagonist).

In another aspect, other cell adhesion inhibitors may also be used alone by itself or by combination with others (Rideway 2003). The list includes but not limited to, the described CXCR4 antagonist, compounds such as proteases, e.g. neutrophil elastase and cathepsin G, which may be able to cleave VCAM-1, MMP-9 related molecules such as IL8, and all compounds that inhibit any chemokine ligand and receptor combinations, e.g. GROβ/CXCR2.

In another aspect, AIM cells preparations will be shown to compose of adequate numbers of AIM cells that contain active immature and mature dendritic like cells with phenotypic characterization done by flow-cytometry consisting of expression patterns in CD80, CD86, CD11c. CD14, CD1a and CCR7 etc. AIM cells also will be shown to compose of adequate number of immune cells such as CD4+, CD8+ T cells and NKT cells.

In one aspect, the priming for the collection of AIM is done by giving an individual, simultaneously or in sequence, with one or a combination of more than one agents from a long list of APC maturing and proliferating agents such as G-CSF, GM-CSF, IL4, IFN, SCF, TNF alpha and prostaglandin E2 etc. (Group A)

In another aspect, the priming before the use of cell adhesion inhibitor and the collection of AIM consists of giving an individual one or more than one Vaccine as described in the Glossary. The Vaccine can be made from irradiated autologous or allogeneic cells with or without the addition of an adjuvant. The Vaccine can be made from peptides, tumor derived mRNA etc. Some of these Vaccines use vectors to make them more effective. Vectors can be special viruses, bacteria, yeast cells, or other structures that can be used to get antigens or DNA into the body. (Vaccine as described in the Glossary)

In yet another aspect, the priming before the use of cell adhesion inhibitor and the collection of AIM consists of giving an individual with one or a combination of more than one agents such as anti-CTLA 4, anti-PD1, TLR ligands, LPS and CD 40 agonist etc. (Group B)

In another aspect, the priming before the use of cell adhesion inhibitor and the collection of AIM consists of giving an individual therapies that will reduce cancer cell numbers, therapies such as chemotherapy, radiation therapy, molecular targeted therapy, and oncolytic viral therapy.

In yet another aspect, the priming before the use of cell adhesion inhibitor and the collection of AIM consists of giving an individual therapies that will reduce Treg or other suppressors, such as minimal to moderate doses of chemotherapy, anti-CD25, glucocorticoid induced tumor necrosis factor (GITR) agonists or ligands, Imatinib, Bevicuzimab and lenalidomide. (Group C)

In yet another aspect, priming before the use of cell adhesion inhibitor and the collection of AIM consists of giving an individual therapies that will amplify the attraction of immune cells to the cancer area, the cross presentation of tumor antigen by APCs and the activation of CD4+ and CD8+ T cells and NKT cells. Therapies well known in this regard are oncolytic viruses with GM-CSF expression (e.g. CG0070) or autologous and allogeneic cancer cell (e.g. GVAX) with gene expression of cytokines such as GM-CSF, IFN, TNF etc.

In one aspect, apheresis is used to isolate AIM from the peripheral blood using known blood cell separators that are now available in the commercial market. Examples are machines made by Haemonetics V50 blood separator, the Baxter CS 3000, the Fresenius AS 104 and the Fresenius AS TEC 104 and the Excel. By varying the separation method, apheresis can be adapted to isolate different cellular components from the peripheral blood. In the case of AIM, the methodology usually employs those methods that separate the mononuclear cell fraction. The final AIM product may be cleansed and further concentrated by density gradient centrifugation in an automatic process and be available for immediate use or be cryogenically stored for later use. Cryogenic preservation methods are well known in the art.

In one aspect, the simple incubation of the cell types in AIM by themselves will enhance the committed immune cells into proliferation and be ready for treatment purposes.

In another aspect, an ex vivo process of incubation of AIM cells with a Vaccine (from the Vaccine shown in Glossary) is performed before being ready for use.

In a further aspect, an ex vivo process of incubation of AIM with Group A agents such as different cytokines, hormones or molecules that can increase interaction and further maturation of APC, immune cells with some of the antigens (e.g. circulating cancer cells that are detached from the tumor or lymph node stroma because of the cell adhesion inhibitor) such as GM-CSF, IL4, IFN, SCF, TNF alpha and prostaglandin E2 etc.

In one aspect, an ex vivo process of incubation of AIM with Group B agents to increase the proliferation of committed immune cells into cell type or antigen specific cytotoxic T cells (CTL) by the addition of agents such as anti-CTLA 4 antibody, anti-PD1 antibody, LPS, TLR ligands, CD40 agonist etc.

In yet another aspect, an ex vivo process of incubation of AIM with Group C agents that will reduce Treg and other suppressor mechanisms, such as minimal to moderate doses of chemotherapy, anti-CD25, glucocorticoid induced tumor necrosis factor (GITR) agonists or ligands, Imatinib, Bevicuzimab, lenalidomide and OX 40 agonist etc.

In another aspect, an ex-vivo process of incubation of AIM with or without Vaccine, together with one or more than one agents from each, both or all of the above groups A, B and C (the group of cytokines, the group of enhancing agents such as anti-CTLA 4 etc., and the group of anti-suppressors such as chemotherapy)

In yet another aspect, the ex-vivo process of incubation of AIM with agents described above from groups A, B and C is being done with dose ranges that are much higher than the maximum tolerable human dose than can be given in vivo. The combination ex-vivo incubation of these super high doses of agents from these groups, with or without Vaccines and tumor antigens may enable the use of AIM therapy in normally immunogenic but resistant (immunologically resistant melanoma, prostate cancer, renal cell carcinoma etc.) and non immunogenic tumors to break tolerance. An example will be given for the use of AIM to treat late stage patients with diseases such as non-small cell lung, colon, ovarian and pancreatic cancer etc.

In another aspect, a kit with a formulation prepared with one or a combination of more than one agents from the Group of Vaccines, Group A, B and C may be formulated and be used in the ex vivo preparation or incubation of the AIM cells. Other suitable additives in the kit with this formulation for administration can include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, and adjuvants.

In another aspect, cells in the AIM cells can further be enriched differentially by those based on surface antigens expressed by certain types of DCs, CD4+ CD8+ T cells, and NKT cells e.g. using FACS so that the fractions of the different kinds of cells in the AIM cells can be altered. Alternatively, cells can be sorted by mixing with magnetic beads coated with monoclonal antibodies against a cell surface antigen characteristically expressed by these cells. In summary, any known methodology in the art can be employed to further change the composition of the different cell types within the AIM cells repertoire.

In another aspect, AIM with or without an ex vivo process, can be used to generate vaccines and part of it to be cryogenically preserved for further treatment in the future. Acceptable adjuvants may also be added to enhance the immunological response. Methods for formulating APC vaccine or a preparation for adoptive immunotherapy are known to those skilled in the art. As a common practice, AIM cells are washed and re-suspended in heat-inactivated plasma (preferably autologous plasma) and 10% dextrose at a concentration of 2×10⁵ cells/ml. The cells can then be diluted 1:1 with a mixture of heat-inactivated plasma and 20% DMSO to give a final concentration of 5% dextrose, 10% DMSO in heat-inactivated plasma. The target final filled formulation is 1 to 10×10⁵ cells/ml. in a container suitable for cyro-preservation. The unused AIM cells can then be frozen at −80 degree C. and thawed before administration in the next few cycles of vaccination. The rest may be stored for long term use in a cryogenic freezer (preferably in a liquid nitrogen freezer designed to prevent contamination), preferably at a temperature of −150 degree C. The frozen vaccine may also be shipped to another clinical site for patient administration immediately upon thawing.

In yet another aspect, AIM preparation, with or without an ex vivo process, can be used as adoptive immunotherapy by the usually routes of administration to an individual with the disease. One of the usual routes for systemic adoptive immunotherapy is by an intravenous infusion. For intravenous infusion, the AIM cells can be placed in acceptable carriers with formulations well known in the art (e.g. Remington's Pharmaceutical Sciences 16^{th} edition, Osol, A. Ed. 1980). The cells are preferably being formulated in a solution with a pH from 6.5 to 8.5. Excipients to bring the cell mixture solution to isotonicity can also be added, such as 4.5% mannitol, normal 0.9% saline or sodium phosphate. Other pharmaceutically acceptable agents can also be added to bring the solution to isotonicity, including, but not limited to dextrose, boric acid, sodium tartrate, propylene glycol or other inorganic or organic solutes.

In one aspect, the use of AIM cells vaccine and AIM cells preparations for adoptive immunotherapy is for the treatment of cancer, including but not exclusively limited to, cancers such as melanoma, prostate cancer, breast cancer, renal cell carcinoma, colon cancer, AML, and CML.

In another aspect, the use of AIM cells vaccine and AIM cells preparations for adoptive immunotherapy is for infectious diseases, including but not limited to, diseases such as HIV.

### EXAMPLES

### General Principles:

### Pre AIM Therapy:

Most established cancer therapy methods are synergistic with AIM tumor immunotherapy. The use of surgery, radiation, chemotherapy, molecular targeted therapy and oncolytic viruses etc. may be helpful to reduce tumor burden, which is always an additive advantage. Increase tumor cell necrosis and the chance of more cell adhesion receptors expressed by tumor cells under those circumstances may enhance tumor cell mobilization and presentation to AIM cells.

### Priming Regimens:

Priming may not be necessary in AIM therapy. If it were to be used, one or a combination of more than one agents from one and/or more than one group from the Groups A, B and C can be chosen as a priming regimen to enhance the effectiveness of the interaction between tumor cells and their antigens with the AIM cells in the previously described micro niche environment prior to cell adhesion inhibitor therapy. The duration, effective dose range of each agent being used should be familiar to those skilled in the art.

### Cell Adhesion Inhibitor:

Patient was given Plerixafor (CXCR4 antagonist) at 0.24 mg/kg by the subcutaneous route at 9 pm on the day prior to apheresis. The timing of 9 pm is to allow about 12 hours before apheresis so that the necessary cell components in AIM are mobilized into the peripheral blood. This time period of 12 hours can be shortened or lengthened accordingly by those familiar with the art for more effective cell mobilization and collection.

### Apheresis:

Apheresis or leukapheresis is done after a suitable time period of cell adhesion inhibitor administration as described in the Glossary.

### AIM Ex-Vivo Culture Methodology:

AIM cells may be used as is by a short incubation as described in Protocol 1 in the Glossary.

The short incubation period may be a very useful process for on-site preparation as no clean room facility is required and the requirement for stringent QC procedures and tests is far less than those that require more than 48 hours of incubation.

Even for short duration incubation, AIM cells may also activated further by a culture method that involves a host of different agents from one or a combination of more than one groups from Group A, B and C as described in Protocol 2 and 3 in the Glossary.

For longer duration incubation, e.g. more than 48 hours, the same agents outlined in Protocol 2 and 3 may be utilized with the necessary GMP qualification of the safety, viable cell count, cell markers, sterility and toxicology profiles etc. being completed before the release of the final product for treatment purpose.

AIM Cell in Combination with Other Form of Immunotherapy:

AIM cell can also be used with other proprietary forms of tumor immunotherapy. An example with Sipuleucel-T immunotherapy is illustrated in Example IV.

### AIM Cell Vaccine or AIM Cell Preparation for Adoptive Immunotherapy:

The decision to use AIM cells in the form of a vaccine to be given as sub-dermal injections against the decision to use AIM cells as an intravenous infusion for adoptive immunotherapy is a delicate one and may depend, speculatively, on the expectation (supported by cell markers and cytokine studies in the AIM cell preparation) of which preparation will have the more dominant and activated portion of effector cells. In general, if the dominant fraction of cells in the AIM cell preparation is expected to be APCs, then vaccination may be more appropriate. Conversely, if the dominant fraction of the AIMs cell preparation is expected to be the immune T cells, then intravenously adoptive immunotherapy may be more appropriate. If both fractions are about equal, then it will be the decision based on the patient's tumor type, physical conditions and other subsequent therapies etc. Similarly, the number of AIM cells given per dose, the frequency of treatment, the timing of treatment in association with other synergistic therapies, the route of administration etc. are all subjected to great variability based on the clinical judgment of those familiar and skilled in the art. It is important to bear in mind the scenario of events and procedures as described below are all just examples.

In conclusion, the actual details of how to use AIM cells for the treatment of cancer and other diseases will vary, albeit the basic principles of practice to use this invention will remain the same.

### Example I

Treatment of Advanced Colorectal Cancer Primed (Stage IV) with Group A Agents and then with Autologous Tumor Lysate (Vaccine) and Anti-CTLA 4 (Group B) in the Ex Vivo Cultured AIM Cells Preparation for Adoptive Immunotherapy

Autologous Tumor Culture:

After surgical resection, the colorectal patient's tumor sample will be processed for tissue culture by mincing them with scissors and passing them through metal meshes of decreasing pore size. The cell suspension will then be plated onto tissue culture flasks and grown in DMEM/F10 (Irvine Scientific, Santa Ana, Calif.) plus 10% FCS (Irvine Scientific) and 1% penicillin/streptomycin (Invitrogen, Carlsbad, Calif.). Tumor culture will be done to ensure that a source be maintained for future testing assessments as well as another way to maintain a new supply source of tumor lysate.

Preparation of Tumor Lysate:

Tumor samples from surgery will have been processed in the laboratory to produce single-cell suspensions as follows: the surgical specimen will be washed thrice in dissection medium (HBSS+30 μg/ml catalase+6.6 μg/ml desferoxamine+25 μg/ml N-acetyl cysteine+94 μg/ml cystine-2HCl+1.25 μg/ml superoxide dismutase+110 μg/ml sodium pyruvate+2.4 μg/ml HEPES+0.36% glucose+800 μM MgCl₂+100 units/ml Fungi-Bact). Then, the specimen will be minced with scissors and pass through metal meshes of decreasing pore size (0.38 and 0.14 mm), followed by a nylon mesh with a pore size of 0.21 mm. Cells will be lysed by four freeze (on liquid nitrogen)/thaw cycles (room temperature). Lysis will be monitored by light microscopy, and larger particles will be removed by centrifugation (1900 rpm for 10 min at 4° C.). Supernatants will then pass through a 0.2 μm filter, the protein concentration will be determined by Bio-Rad protein assay, and aliquots will be frozen at −80° C. until use.

Priming of Patients with Growth Factors:

Patient will be given GM-CSF at a dosage of 10 μg/kg by subcutaneous route per day for 7 days.

In the second phase of this priming, the patient will be given interferon β1a at a dosage of 44 μg/kg by the subcutaneous route on days 5 and day 7.

Cell Adhesion Inhibitor Administration:

Patient will be given Plerixafor (CXCR4 antagonist) at 0.24 mg/kg by the subcutaneous route at 9 pm on day 7.

AIM Cells Preparation:

On day 8, at or around 9 am, mononuclear cells will be isolated and collected by leukapheresis. A Fenwal CS 3000 blood cell separator is used to harvest the mononuclear cell layer. Leukapheresis usually yields about 1-100 (usually 2-20)×10⁶ AIM cells. The exact AIM cells cell numbers in the product pack will be counted and 1-4 aliquots of 5×10⁶AIM cells each will be transferred into separate bags using aseptic technique and stored at a temperature of −80 degree C. Some of the AIM cells are immediately taken to short or long incubation while the rest will be cryo-preserved under standard protocol. The exact cell count will vary a great deal pending on the priming agents and duration of each cycle of therapy.

AIM cells Phenotypic and Cytokines Evaluation:

AIM cells will be re-suspended in PBS containing 2% fetal bovine serum (v/v) and stained with anti-CD14 FITC, anti-HLADR phycoerythrin (PE), and biotinylated anti-CD1a, CD-1d, TCRαβ, CD-4, CD-8, CD69 (T cell activation marker), CD80, CD83, CD86, anti-CD54, anti-CD40, antibodies (BD PharMingen). Evaluation of IFNαa, IFNγ, IL4, IL1, IL6, TNFα, TGFβ, IL10 and IL12 expression is done whenever it is appropriate to do so. Evaluation of suppressor cell such as Treg i.e., CD4+ CD25+ foxp3+ cells and other myeloid suppressors and Tr1 and Th3 cell markers expression is also done whenever it is appropriate to do so. The evaluation of different subsets of dendritic cells, other APCs, helper and suppressor T cells, B cells and NKT cells are well known to those familiar and skilled in the art.

Culture of AIM cells with Autologous Tumor Lysate and Anti-CTLA 4:

On the day before each of the three AIM cells infusions (days −1, 13, and 27), one aliquot each of AIM cells containing 5×10⁶ cells will be washed in RPMI 1640 with autologous patient serum supplemented with one aliquot of tumor lysate together with 0.04 ug/ml of anti-CTLA 4. The AIM cellss with the tumor lysate and anti-CTLA 4 will be incubated overnight for 18 h at 37° C. on a tissue rotator.

Treatment Schedule:

AIM cells intravenous infusions will be administered three times on days 0, 14 and 28.

Note: As described earlier, the dose of medication and agents used will be in the effective dose range in the context of this invention and should be well known to those skilled in the art.

### Example II

Treatment of Bladder Cancer Patient Pre Radical Cystectomy using an Oncolytic Virus Expressing GM-CSF Given by the Intravesical Route and AIM Cells as Vaccine Post Operatively

Pre Treatment and Priming:

Patient with bladder cancer stage I to IV who is scheduled for radical cystectomy is eligible to received two doses of oncolytic adenovirus with GM-CSF expression (e.g. CG 0070) at one week apart. Dose level of CG0070, e.g. is 1×10¹² viral particles per treatment given by an intravesical instillation.

Radical cystectomy and lymph nodes resection will be done four to six weeks (or more depending on patient physical condition) after the last dose of CG 0070 intra vesical instillation and AIM cell vaccination.

Cell Adhesion Inhibitor Administration:

Patient will be given Plerixafor (CXCR4 antagonist) at 0.24 mg/kg by the subcutaneous route at 9 pm on the day prior to apheresis, and one week after the day of intravesical instillation of CG0070. This is repeated again 7 days after the second intravesical instillation of CG 0070 about one month later.

AIM Cells Preparation:

On the day of apheresis (see Glossary for detail procedure), at or around 9 am, mononuclear cells will be isolated and collected by leukapheresis. A Fenwal CS 3000 blood cell separator is used to harvest the mononuclear cell layer. Leukapheresis yielded about 1-100×10⁶ AIM cells. The exact AIM cells cell numbers in the product pack was counted and 1-5 aliquots of 5×10⁶ AIM cells each were transferred into separate bags using aseptic technique and stored at a temperature of −80 degree C. Some of the AIM cells are immediately taken to short or long incubation while the rest will be cryo-preserved under standard protocol. The exact cell count will vary a great deal pending on the priming agents and duration of each cycle of therapy.

AIM cells Vaccine Phenotypic and Cytokines Evaluation:

AIM cells will be resuspended in PBS containing 2% fetal bovine serum (v/v) and stained with anti-CD14 FITC, anti-HLADR phycoerythrin (PE), and biotinylated anti-CD1a, CD-1d, TCRαβ, CD-4, CD-8, CD69, CD80, CD83, anti-CD86, anti-CD54, anti-CD40 antibodies (BD PharMingen). Evaluation of IFNα, IFNγ, IL4, IL1, IL6, TNFα, TGFβ, IL10 and IL12 expression is done whenever it is appropriate to do so. Evaluation of suppressor cell such as Treg, CD4+ CD25+ foxp3+ cells and other myeloid suppressors, Tr1 and Th3 cell markers expression is also done whenever it is appropriate to do so. The evaluation of different subsets of dendritic cells, other APCs, helper and suppressor T cells, B cells and NKT cells are well known to those familiar and skilled in the art.

Culture of AIM Cells with Anti-CTLA 4:

On the day before each of the three AIM cells vaccinations (days 1, 15, 29), one aliquot each of AIM cells diluted to 5×10⁶ cells will be washed in RPMI 1640 with autologous patient serum supplemented with 0.04 ug/ml of anti-CTLA 4. The AIM cells with anti-CTLA 4 will be incubated for 6 hours at 37° C. on a tissue rotator before use.

AIM Cells Vaccination and Radical Cystectomy:

The patient will receive 5×10⁶ AIM cells vaccine cells intradermally (i.d.) in the upper leg, 5-10 cm from an inguinal lymph node. The schedule of a total of three bi-weekly, starting one day after apheresis, intradermal vaccinations and a repeat cycle of intra vesical instillation of CG0070, followed 7 days later by another series of apheresis and three bi-weekly AIM cell vaccination. Radical cystectomy will be performed four to six weeks after the last intravesical CG 0070.

Note: As described earlier, the dose of medication and agents used will be in the effective dose range in the context of this invention and should be well known to those skilled in the art.

### Example III

Treatment of Pancreatic Cancer with Autologous or Allogeneic Cell Vaccine Expressing GM-CSF (e.g GVAX) with Low Dose Cyclophosphamide and AIM Cells Adoptive Immunotherapy

Pre treatment and Priming: All patients will undergo extensive surgical resection of their tumors. The GVAX cell vaccine is being administered as an intradermal injection before and after standard postoperative adjuvant radiation therapy and fluorouracil chemotherapy. Patients will receive up to five doses—the first prior to adjuvant chemoradiotherapy, the next three following adjuvant therapy at approximately 1-month intervals, and the fifth as a booster injection 6 months later. Priming with a low dose cyclophosphamide of 500 mg orally as an anti-suppressor cell therapy with each GVAX vaccination (Group C agents).

Cell Adhesion Inhibitor Administration:

Patient will be given Plerixafor (CXCR4 antagonist) at 0.24 mg/kg by the subcutaneous route at 9 pm 7 days after each GVAX vaccination.

AIM Cells Preparation:

On day 8 after each GVAX vaccination, at or around 9 am, mononuclear cells will be isolated and collected by leukapheresis. A Fenwal CS 3000 blood cell separator is used to harvest the mononuclear cell layer. Leukapheresis yielded about 1-100×10⁶ AIM cells. The exact AIM cells cell numbers in the product pack was counted and 1-5 aliquots of 5×10⁶ AIM cells each were transferred into separate bags using aseptic technique and stored at a temperature of −80 degree C. Some of the AIM cells are immediately taken to short or long incubation while the rest will be cryo-preserved under standard protocol. The exact cell count will vary a great deal pending on the priming agents and duration of each cycle of therapy.

AIM Cells Vaccine Phenotypic and Cytokines Evaluation:

AIM cells will be resuspended in PBS containing 2% fetal bovine serum (v/v) and stained with anti-CD14 FITC, anti-HLADR phycoerythrin (PE), and biotinylated anti-CD1a, CD-1d, TCRαβ, CD-4, CD-8, CD69 (T cell activation marker), CD80, CD83, anti-CD86, anti-CD54, anti-CD40 antibodies (BD PharMingen). Evaluation of IFNα, IFNγ, IL4, IL1, IL6, TNFα, TGFβ, IL10 and IL12 expression is done whenever it is appropriate to do so. Evaluation of suppressor cell such as Treg, CD4+ CD25+ foxp3+ cell and other myeloid suppressors, Tr1 and Th3 cell markers expression is also done whenever it is appropriate to do so. The evaluation of different subsets of dendritic cells, other APCs, helper and suppressor T cells, B cells and NKT cells are well known to those familiar and skilled in the art.

Culture of AIM cells with anti-CTLA 4:

On the day before each of the AIM cells infusions (maximum five depending on the GVAX vaccinations), AIM cells containing at least 5×10⁶ cells (use most of the aliquots available) will be washed in RPMI 1640 with autologous patient serum supplemented with 0.04 ug/ml of anti-CTLA 4. The AIM cells and anti-CTLA 4 will be incubated for 6 hr at 37° C. on a tissue rotator.

Treatment Schedule:

AIM cells intravenous infusions will be administered on the 8^{th} day after each GVAX vaccination.

Note: As described earlier, the dose of medication and agents used will be in the effective dose range in the context of this invention and should be well known to those skilled in the art.

### Example IV

Treatment of Metastatic Castration Resistant Prostate Cancer by Priming the Patient with an Oncolytic Virus Targeting PSA Positive Cancer Cells (e.g CG7870) Leading to AIM Cells and Ex Vivo Recombinant Fusion Protein PA2024 (Prostate Antigen, Prostatic Acid Phosphatase Fused to GM-CSF) Known as Sipuleucel-T Immunotherapy

Priming:

Priming is done by the Intra tumor injection of a prostatic specific adenovirus construct CG7870 (PSA control expression of the E1A gene and retaining the E3 gene for high tumor potency) at 1×10⁷ viral particles per mm² of tumor 7 days prior to apheresis. This injection of CG7870, apheresis and the generation of AIM are being repeated every two weeks for three, and then another session to be repeated three months later if no disease progression.

Cell Adhesion Inhibitor Administration:

Patient will be given Plerixafor (CXCR4 antagonist) at 0.24 mg/kg by the subcutaneous route at 9 pm one day prior to apheresis and 7 days after each CG7870 intra tumor injections.

AIM Cells Preparation:

On the day of ahperesis, at or around 9 am, mononuclear cells will be isolated and collected by leukapheresis. A Fenwal CS 3000 blood cell separator is used to harvest the mononuclear cell layer. Leukapheresis yielded about 1-100×10⁶ AIM cells. The exact AIM cells cell numbers in the product pack was counted and 1-5 aliquots of 5×10⁶ AIM cells each were transferred into separate bags using aseptic technique and stored at a temperature of −80 degree C. Some of the AIM cells are immediately taken to short or long incubation while the rest will be cryo-preserved under standard protocol. The exact cell count will vary a great deal pending on the priming agents and duration of each cycle of therapy.

AIM cells Vaccine Phenotypic and Cytokines Evaluation:

AIM cells will be resuspended in PBS containing 2% fetal bovine serum (v/v) and stained with anti-CD14 FITC, anti-HLADR phycoerythrin (PE), and biotinylated anti-CD1a, CD-1d, TCRαβ, CD-4, CD-8, CD69 (T cell activation marker), CD80, CD83, anti-CD86, anti-CD54, anti-CD40 antibodies (BD PharMingen). Evaluation of IFNα, IFNγ, IL4, IL1, IL6, INFα, TGFβ, IL10 and IL12 expression is done whenever it is appropriate to do so. Evaluation of suppressor cell such as Treg, CD4+ CD25+ foxp3+ cell and other myeloid suppressors, Tr1 and Th3 cell markers expression is also done whenever it is appropriate to do so. The evaluation of different subsets of dendritic cells, other APCs, helper and suppressor T cells, B cells and NKT cells are well known to those familiar and skilled in the art.

Culture of AIM cells with Sipuleucel-T:

Ex vivo culture of AIM cells will be performed under the Sipuleucel-T protocol of incubation with PA2024 for 36 to 44 hours at 37 degree C. The cells will be washed before final formulation.

Administration:

Patients will receive AIM cells Sipuleucel-T intravenously during a period of 60 minutes for three treatments every two weeks. The treatment will continue for any session of three treatments every two weeks three months later if disease does not progress.

### Example V

Treatment of Metastatic Lung Cancer with a Priming of Anti-PD1, Low Dose Chemotherapy and the Generation of AIM Cells with Anti-CTLA 4 for Adoptive Immunotherapy

Priming:

Metastatic lung patients that have failed chemotherapy will be enrolled and be given anti-PD1 at a dose range of 3-10 mg/kg per dose given intravenously with an oral low dose cyclophosphamide of 500 mg. 7 days prior to apheresis and AIM cells therapy.

Cell Adhesion Inhibitor Administration:

Patient will be given Plerixafor (CXCR4 antagonist) at 0.24 mg/kg by the subcutaneous route at 9 pm 7 days after each anti-PD1 and cyclophosphamide administration.

AIM Cells Preparation:

On day 8 after each anti-PD1 and cyclophosphamide administration, at or around 9 am, mononuclear cells will be isolated and collected by leukapheresis. A Fenwal CS 3000 blood cell separator is used to harvest the mononuclear cell layer. Leukapheresis yielded about 1-100×10⁶ AIM cells. The exact AIM cells cell numbers in the product pack was counted and 1-5 aliquots of 5×10⁶AIM cells each were transferred into separate bags using aseptic technique and stored at a temperature of −80 degree C. Some of the AIM cells are immediately taken to short or long incubation while the rest will be cryo-preserved under standard protocol. The exact cell count will vary a great deal pending on the priming agents and duration of each cycle of therapy.

AIM Cells Vaccine Phenotypic and Cytokines Evaluation:

AIM cells will be resuspended in PBS containing 2% fetal bovine serum (v/v) and stained with anti-CD14 FITC, anti-HLADR phycoerythrin (PE), and biotinylated anti-CD1a, CD-1d, TCRαβ, CD-4, CD-8, CD69 (T cell activation marker), CD80, CD83, anti-CD86, anti-CD54, anti-CD40 antibodies (BD PharMingen). Evaluation of IFNα, IFNγ, IL4, IL1, IL6, TNFα, TGF6, IL10 and IL12 expression is done whenever it is appropriate to do so. Evaluation of suppressor cell such as Treg, CD4+ CD25+ foxp3+ cell and other myeloid suppressors, Tr1 and Th3 cell markers expression is also done whenever it is appropriate to do so. The evaluation of different subsets of dendritic cells, other APCs, helper and suppressor T cells, B cells and NKT cells are well known to those familiar and skilled in the art.

Culture of AIM Cells with Anti-CTLA 4:

AIM cells containing at least 5×10⁶ cells (use most of the aliquots available) will be washed in RPMI 1640 with autologous patient serum supplemented with 0.04 ug/ml of anti-CTLA 4. The AIM cells and anti-CTLA 4 will be incubated for 6 hr at 37° C. on a tissue rotator.

Treatment Schedule:

AIM cells intravenous infusions will be administered on the 8^{th} day after each anti-PD1 and cyclophosphamide administration. The whole cycle will be repeated every two weeks until tumor progression.

Note: As described earlier, the dose of medication and agents used will be in the effective dose range in the context of this invention and should be well known to those skilled in the art.

### Example VI

Protocol of AIM Cells Preparation to Treat Late Stage Solid Tumors by Ex Vivo Incubation with Super High Dose Agents

Priming of Patients with Group A Agents (Optional):

Patient may (if tolerable) be given GM-CSF at a dosage of 10 μg/kg by subcutaneous route per day for 5 days.

Cell Adhesion Inhibitor Administration:

Patient will be given Plerixafor (CXCR4 antagonist) at 0.24 mg/kg by the subcutaneous route at 9 pm on day 5.

AIM Cells Preparation:

On day 6, at or around 9 am, mononuclear cells will be isolated and collected by leukapheresis. A Fenwal CS 3000 blood cell separator is used to harvest the mononuclear cell layer. Leukapheresis usually yields about 1-100 (usually 2-20)×10⁶ AIM cells. The exact AIM cells cell numbers in the product pack will be counted and 1-3 aliquots of 5×10⁶AIM cells each will be transferred into separate bags using aseptic technique and stored at a temperature of −80 degree C. Some or all of the AIM cells are immediately taken to short or long incubation while the rest will be cryo-preserved under standard protocol. The exact cell count will vary a great deal pending on the priming agents and duration of each cycle of therapy.

AIM Cells Vaccine Phenotypic and Cytokines Evaluation:

AIM cells will be resuspended in PBS containing 2% fetal bovine serum (v/v) and stained with anti-CD14 FITC, anti-HLADR phycoerythrin (PE), and biotinylated antiCD1a, CD-1d, TCRαβ, CD-4, CD-8, CD69 (T cell activation marker), CD80, CD83, anti-CD86, anti-CD54, anti-CD40 antibodies (BD PharMingen). Evaluation of IFNα, IFNγ, IL4, IL1, IL6, TNFα, TGFβ, IL10 and IL12 expression is done whenever it is appropriate to do so. Evaluation of suppressor cell such as Treg, CD4+ CD25+ foxp3+ cell and other myeloid suppressors, Tr1 and Th3 cell markers expression is also done whenever it is appropriate to do so. The evaluation of different subsets of dendritic cells, other APCs, helper and suppressor T cells, B cells and NKT cells are well known to those familiar and skilled in the art.

Culture of AIM Cells with Super High Dose Agents:

On day 6, after the collection of AIM cells, one to all of aliquots of AIM cells containing 5×10⁶ cells will be washed in RPMI 1640 with autologous patient serum supplemented with a mixture of super high doses (10 to 1000 times of the pharmaceutical effective dose) of agents selected from one or a combination of more than one in Groups A, B and C and the Vaccine Group. The AIM cells will be incubated overnight for 2-48 hr. at 37° C. on a tissue rotator. A long incubation period of over 48 hours may be necessary. The cells will be re-washed in RPMI 1640 to get rid of all residual agents before use as the final AIM cells preparation

Treatment Schedule:

The final AIM cells preparation will be given by intravenous infusions as adoptive immunotherapy after the ex vivo incubation and cleansing. The treatment cycle will be repeated every two weeks until disease progression.

### BIBLIOGRAPHY

Banchereau. “Melanoma APC preparation.” Cancer Research 61 (2001): 6451-58.
Belz. “The CD8a+APCs is responsible for inducing peripheral self intolerance to tissue associated antigens.” J. Exp. Med, 2002: 1099-1104.
Blankenstein. “Cross priming versus cross tolerance: are two signals enough?” Trends in Immunol, 2002: 171-173.
Fanutuzzi. “Loss of CCR2 expression and functional response to monocyte chemotactic protein MCP-1 during the differentiation of human AIM cells.” Blood, 1999: 875-883.
Gabriele. “IFN alpha promotes differentiation of AIM cells from patients with CML into activated APCs mature after LPS.” Blood, 2004: 980-987.
Kaiser. “Naive CD8+ T cells recruitment and proliferation are dependent on stage of APCs maturation.” Eur. J Immunol, 2003: 162-171.
Kryczek. “Stroma-derived factor (SDF-1/CXCL12) and human pathogenesis” Am J Physiol Cell Physiol 292: C987-C995, 2007
Lapenta. “Potent immune response against HIV-1 and protection from virus challenge in hu-PBL-SCID mice immunized with inactivated virus pulsed APCs generated in the presnece of IFN-alpha.” J Exp Med, 2003: 361-367.
LeBon. “Links between innate and adaptive immunity via type 1 interferon.” Curr. Opin Immunology, 2002: 432-436.
Melief. “Cancer Immunotherapy by Dendritic Cells” Immunity 29, Sep. 19, 2008 372-383
Oppenheim. “Chemokine receptors on dendritic cells promote autoimmune reactions” Arthritis Res 2002, 4 (supp 3): S183-S188
Rideway. “APC vaccine or a preparation for adoptive immunotherapys (response rate).” Cancer Investigations 21 (2003): 873-876.
Ridge. “A conditioned APCs can be a temporal bridge between a CD4+T-helper and a T-killer cell.” Nature, 1998: 474-478.
Santini. “Type I interferon as a powerful adjuvant for monocyte derived APC development and activity in vitro and in HuPBL-SCID mice.” J. Exp. Med, 2000: 1777-1788.

Santini, Lapenta and. “IFN alpha conditioned APCs efficient in cross priming CD8+ cells against viral antigens.” Eur J Immunol, 2006: 2046-2060.
Santodonato. “Monocyte derived APCs generated after a short term culture with IFN and GMCSF stimulate a potent EBV specific CD8+ response.” J Immunol, 2003: 5195-5202.
Schuler. “Use of APCs in cancer immunotherapy.” Curr. Opon Immunol, 2003: 138-147.
Small. “Provenge Survival.” Dendreon 09001 and 9002. trials, 2007.
Yeung, Alex W. “Double cycle high dose chemotherapy with peripheral blood stem cell and hematopoietic growth factor support in patients with advanced solid tumor.” Cancer 73, no. 7 (1994): 1960-1970.

The claims are:

1. A method of using one or a combination of more than one type of cell adhesion inhibitors such as Plerixafor (CXCR4 antagonist) to mobilize and then collect from the peripheral blood of an individual a mixture of Antigen Presenting Cells (APCs and which would include all types of Dendritic Cells or DCs) and Immune Cells (which would include CD4+, CD8+ T cells and NKT cells) to be named as the APCs and Immune Cells Mixture (AIM).

2. The method of claim 1, wherein the cell adhesion inhibitors can be any one or a combination of more than one from agents such as proteases, e.g. neutrophil elastase and cathepsin G (VCAM-1 cleavage), MMP-9 related molecules such as IL8, other molecules that can inhibit any chemokine ligand and receptor combinations, e.g GROβ/CXCR2.

3. The method of claim 1, wherein the cell adhesion inhibitor mobilization and AIM cells preparation may or may not consist of a priming procedure (see definition of Priming in the Glossary) using one agent or a combination of more than one agents from Group or Groups that have specific enhancement of the therapeutic actions of AIM cells preparation.

4. The method of claim 3, wherein the Group can be drugs, physical means, molecules, or viral particles that are defined as chemotherapeutic, radiation, molecular targeting or oncolytic agents, or more than likely, cancer chemotherapeutic, cancer radiation therapy, cancer molecular targeting and cancer cell specific oncolytic viral agents.

5. The method of claim 3, wherein the Group can be molecules that are defined as cytokines, immune related or pro-inflammatory agents such as G-CSF, GM-CSF, IL4, IFN, SCF (Steele factor, Stem Cell Factor kit ligand), TNFα (tumor necrosis factor α), prostaglandin E2, IL1, IL6 etc. (Group A in Glossary)

6. The method of claim 3, wherein the Group can be agents that are defined as anti down-regulate or up-regulate immune cell agents such as anti-CTLA 4, anti-PD 1, CD 40 agonist, LPS, TLR ligands etc. (Group B in Glossary)

7. The method of claim 3, wherein the Group can be agents that are defined as anti suppressors or anti-Treg agents such as low dose chemotherapeutic agents, e.g. cyclophosphamide, gemcitabine, anti-CD 25, GITR agonist and OX40 agonist etc. (Group C in Glossary)

8. The method of claim 3, wherein the Group can be defined as a Vaccine (see specific definition of Vaccine in Glossary) that may include irradiated autologous or allogeneic tumor cells, tumor lysates, shred tumor vaccine, apoptotic tumor cells, known tumor specific or associated antigens or peptides such as Mage-1, Mage-3, gp-100, NUC-1 etc., dendritic cell tumor hybrids, tumor derived DNA and RNA, modified cell vaccines such as GVAX, oncolytic agents or vectors expressing different genes and gene products such as GM-CSF, type 1 IFN etc. and any other means or methodology of expressing or presenting antigens to an individual that are well known to those skilled in the art.

9. The method of claim 1, wherein the cell adhesion inhibitor mobilization and AIM cells preparation is performed after a priming (see definition of Priming in the Glossary) procedure that uses agents as defined in Groups A, B and C as local and regional therapies such as intra-arterial injections, intra-tumoral injections, intra-vesical perfusions and any other practice of local and regional therapies that are well known to those skilled in the art.

10. The method of claim 3, wherein the cell adhesion inhibitor mobilization and AIM cells preparation that may or may not consist of a priming procedure (see definition of Priming in the Glossary) will undergo an ex vivo culture methodology (see Glossary) using none, one or a combination of more than one agent from Group or Groups that have specific enhancement effects on the final AIM cell product.

11. The method of claim 10, wherein the Group can be molecules that are defined as cytokines, immune related or pro-inflammatory agents such as G-CSF, GM-CSF, IL4, IFN, SCF (Steele factor, Stem Cell Factor kit ligand), TNFα (tumor necrosis factor a), prostaglandin E2, IL1, IL6 etc. (Group A in Glossary)

12. The method of claim 10, wherein the Group can be agents that are defined as anti down-regulate or up-regulate immune cell agents such as anti-CTLA 4, anti-PD 1, CD 40 agonist, LPS, TLR ligands etc. (Group B in Glossary)

13. The method of claim 10, wherein the Group can be agents that are defined as anti suppressors or anti-Treg agents such as low dose chemotherapeutic agents, e.g. cyclophosphamide, gemcitabine, anti-CD 25, GITR agonist and OX40 agonist etc. (Group C in Glossary)

14. The method of claim 10, wherein the Group can be defined as a Vaccine (see specific definition of Vaccine in Glossary) that may include irradiated autologous or allogeneic tumor cells, tumor lysates, shred tumor vaccine, apoptotic tumor cells, known tumor specific or associated antigens or peptides such as Mage-1, Mage-3, gp-100, NUC-1 etc., dendritic cell tumor hybrids, tumor derived DNA and RNA, modified cell vaccines such as GVAX, oncolytic agents or vectors expressing different genes and gene products such as GM-CSF, type 1 IFN etc., proprietary products such as Sipuleucel-T Immunotherapy agent, and any other means and methodology of expressing or presenting antigens to an individual that are well known to those skilled in the art.

15. The method of claim 10, wherein the dose or concentration of agents used as ex vivo incubation with AIM cells in the Groups A, B, C and Vaccine is of a magnitude of 10 to 1000 times (or more) more than the maximum physiologically or pharmaceutically effective dose allowed for each agents to be used in humans.

16. The method of claim 10, wherein the ex vivo culture methodology uses a short incubation time of 0-48 hours.

17. The method of claim 10, wherein the ex vivo culture methodology uses a long incubation time of from over 48 hours to 14 days or more.

18. The method of claim 10, wherein the AIM cell final product can further be enriched differentially by those based on surface markers into different cell types such as dendritic cells with different cell markers such as CD11c+ and CD14+, immune cells such as CD4+ and CD8+ T cells and NKT cells using FACS, magnetic beads or other technology known to those skilled in the art.

19. The method of claim 10, wherein the ex vivo culture methodology uses a kit or a formulation that contains the agent or agents used for ex-vivo culture methodology with or without additives suitable for this procedure, additives such as aqueous isotonic sterile injections, antioxidants, buffers, bacteriostats, solutes, adjuvants, serum and culture medium etc.

20. The method of claim 10, wherein the final AIM cell product is used for the preparation of a vaccine to be given by intradermal injections (usual route, other routes may apply) or for the preparation of an intravenous solution (usual route, other routes may apply) for adoptive immunotherapy as described in the examples given.

21. The method of claim 10, wherein the final AIM product is for the treatment of cancer, including but not exclusively limited to, cancers such as melanoma, prostate cancer, breast cancer, renal cell carcinoma, colon cancer, lung cancer, liver cancer, ovarian cancer, head and neck cancer, AML, CML etc.

22. The method of claim 10, wherein the final AIM product is for the treatment of infectious diseases such as HIV etc.
